(19) United States
(12) Patent Application Publication
(10) Pub. No.: US 2026/0210946 A1
(43) Pub. Date: Jul. 23, 2026

(54) METHODS FOR ASSESSING VIABILITY OF DONOR ORGANS THAT UNDERGO PERFUSION

(71) Applicant: The Johns Hopkins University, Baltimore, MD (US)

(72) Inventor: Chirag Parikh, Lutherville, MD (US)

(21) Appl. No.: 19/134,910

(22) PCT Filed: Dec. 4, 2023
(86) PCT No.: PCT/US2023/082270

§ 371 Date: Jun. 2, 2025

Related U.S. Application Data
(60) Provisional application No. 63/429,718, filed on Dec. 2, 2022.

(51) Int. Cl. G01N 33/53

(52) U.S. Cl. CPC G01N 33/5308; G01N 2570/00; G01N 2800/52

(57) ABSTRACT

The present invention relates to the field of organ transplantation. More specifically, the present invention provides compositions and methods useful for assessing viability of donor organs that undergo perfusion. In one embodiment, a method for assessing viability of a donor organ undergoing perfusion comprises the steps of (a) measuring levels of a panel of metabolites comprising one or more of alpha-ketoglutarate (AKG), 3-carboxy-4-methyl-5-propyl-2-furanpropanoate (CMPF), 1-carboxyethylphenylalanine, 1-palmitoyl-2-docosahexaenoyl-glycerophosphocholine (GPC), 1-palmitoyl-2-dihomo-linolenoyl-GPC, and 1-stearoyl-2-docosahexaenoyl-GPC, from a first sample obtained from the organ at the beginning of perfusion; and (b) measuring levels of the panel of metabolites from a second sample obtained from the organ at or near the end of perfusion, wherein levels of the metabolites above a reference indicates that the donor organ is not viable, and wherein levels of the metabolites below the reference indicates that the donor organ is viable.

### CROSS-REFERENCE TO RELATED APPLICATIONS

This application claims the benefit of U.S. Provisional Application No. 63/429,718, filed Dec. 2, 2022, which is incorporated herein by reference in its entirety.

### GOVERNMENT SUPPORT CLAUSE

This invention was made with government support under grant no. DK093770, awarded by the National Institutes of Health. The government has certain rights in the invention.

### FIELD OF THE INVENTION

The present invention relates to the field of organ transplantation. More specifically, the present invention provides compositions and methods useful for assessing viability of donor organs that undergo perfusion.

### BACKGROUND OF THE INVENTION

Hypothermic machine perfusion (HMP) is commonly used in the process of deceased donor kidney transplantation to preserve the organ during transport and help minimize cold ischemic injury to the allograft. HMP circulates cold perfusate solution through the graft kidney for several hours after removal from the donor and before transplantation into the recipient, and there is evidence that HMP reduces delayed graft function (DGF) and improves 3-year graft survival compared with static cold storage (SCS).¹⁻³ However, the biological mechanisms that underlie how HMP contributes to improved kidney graft health are unclear, with some studies suggesting possible roles for downregulation of inflammatory pathways and upregulation of cell survival pathways.^{4,5} Furthermore, while some perfusate proteins have demonstrated moderate predictive ability for delayed graft function, no biomarkers have been associated with graft survival yet.⁶

The need for non-invasive biomarkers to assess allograft viability is greater than ever as both the fraction of kidneys pumped and discarded increases.^{7,8} Even more recently, the change in the kidney allocation system to prioritize candidates within a distance of 250 nautical miles of the donor hospital has led to longer cold ischemia time and increased donor kidney donor profile indices (KDPIs).^{9,10} Given that marginal kidneys are more likely to be pumped, HMP use for organ preservation will likely further increase.¹¹ However, the lack of standardized guidelines for HMP use has led to significant variability among organ procurement organizations (OPOs) in the selection of organs for HMP and the use of biomarkers and other parameters to monitor perfusion. Following the logistical challenges associated with its implementation, approximately 30% of perfused kidneys nationally are later discarded due to concerns about organ quality and viability.¹²

### SUMMARY OF THE INVENTION

Although hypothermic machine perfusion (HMP) is associated with improved kidney graft viability and function, the underlying biological mechanisms are unknown. Untargeted metabolomic profiling may identify potential metabolites and pathways that can help assess allograft viability and contribute to organ preservation. In this multicenter study, the present inventor measured all detectable metabolites in perfusate samples collected at the beginning and end (baseline and post-HMP) of HMP of deceased-donor kidneys, then evaluated their associations with graft failure. In the present inventor's cohort of 190 kidney transplants, 33 (17%) had death-censored graft failure (dcGF) over a median follow-up of 5.0 (IQR: 3.0-6.1) years. The present inventor identified 553 known metabolites in perfusate and characterized their experimental and biological consistency through blind duplicates and unsupervised clustering.

After perfusion-time adjustment and false discovery correction, 6 metabolites in post-HMP perfusate were significantly associated with dcGF: alpha-ketoglutarate, CMPF, 1-carboxyethylphenylalanine, and three glycerol-phosphatidylcholines. All 6 metabolites were associated with an increased risk of graft failure (hazard ratios (HRs) per median absolute deviation ranging 1.04-1.45). Four of the six metabolites also demonstrated significant interaction with donation after cardiac death with notably greater risk in the donation after cardiac death group, with HRs up to 1.69. Discarded kidneys did not have significantly different levels of any dcGF-associated metabolites. On interrogation of pathway analysis, production of reactive oxygen species and increased metabolism of fatty acids were upregulated in kidneys that subsequently developed dcGF. Understanding the role of these metabolites may inform the HMP process and help improve the objective evaluation of allograft offers, thereby reducing the discard of potentially viable organs.

Accordingly, the present invention is based, at least in part, on the development of metabolite biomarkers to assess viability of donor organs that undergo perfusion. In one embodiment, a method for assessing viability of a donor organ undergoing perfusion comprises the steps of (a) measuring levels of a panel of metabolites comprising one or more of alpha-ketoglutarate (AKG), 3-carboxy-4-methyl-5-propyl-2-furanpropanoate (CMPF), 1-carboxyethylphenylalanine, 1-palmitoyl-2-docosahexaenoyl-glycerophosphocholine (GPC), 1-palmitoyl-2-dihomo-linolenoyl-GPC, and 1-stearoyl-2-docosahexaenoyl-GPC, from a first sample obtained from the organ at the beginning of perfusion; and (b) measuring levels of the panel of metabolites from a second sample obtained from the organ at or near the end of perfusion, wherein levels of the metabolites above a reference indicates that the donor organ is not viable, and wherein levels of the metabolites below the reference indicates that the donor organ is viable.

In a specific embodiment, the beginning of perfusion comprises within about ten minutes of the start of perfusion. In another embodiment, at or near the end of perfusion comprises just prior to transfer of the donor organ from an organ procurement organization (OPO) to the recipient medical center. In an alternative embodiment, at or near the end of perfusion comprises just after transfer of the donor organ from an OPO to the recipient medical center.

In particular embodiments, a method for identifying a donor organ as viable or not viable for transplantation comprises the steps of (a) measuring levels of a panel of metabolites comprising one or more of AKG, CMPF, 1-carboxyethylphenylalanine, 1-palmitoyl-2-docosahexaenoyl-GPC, 1-palmitoyl-2-dihomo-linolenoyl-GPC, and 1-stearoyl-2-docosahexaenoyl-GPC, from a sample obtained from an ex vivo organ undergoing perfusion; (b) comparing the measured metabolite levels with the levels of the same metabolites in a reference or control that indicate organ dysfunction or function; and (c) identifying the donor organ as viable when the measured metabolite levels are below the reference levels and identifying the donor organ as not viable when the measured metabolite levels are above the reference levels.

In certain embodiments, a method for transplanting a donor organ that has undergone perfusion comprises the steps of (a) measuring levels of a panel of metabolites comprising one or more of AKG, CMPF, 1-carboxyethylphenylalanine, 1-palmitoyl-2-docosahexaenoyl-GPC, 1-palmitoyl-2-dihomo-linolenoyl-GPC, and 1-stearoyl-2-docosahexaenoyl-GPC, from a sample obtained from an ex vivo organ undergoing perfusion; and (b) transplanting the donor organ to a recipient when the measured levels are below a reference level of the metabolites that indicates organ dysfunction.

In particular embodiments, the donor organ comprises a liver, kidney, heart, pancreas, small intestine, limb, extremity, or a portion of any of the foregoing. In some embodiments, the sample comprises perfusion solution from the organ, biopsy from the organ or a fluid produced by the organ.

In one embodiment, the metabolite panel comprises AKG and one or more of CMPF, 1-carboxyethylphenylalanine, 1-palmitoyl-2-docosahexaenoyl-GPC, 1-palmitoyl-2-dihomo-linolenoyl-GPC, and 1-stearoyl-2-docosahexaenoyl-GPC. In another embodiment, the panel comprises AKG, CMPF, and one or more of 1-carboxyethylphenylalanine, 1-palmitoyl-2-docosahexaenoyl-GPC, 1-palmitoyl-2-dihomo-linolenoyl-GPC, and 1-stearoyl-2-docosahexaenoyl-GPC. In yet another embodiment, the panel comprises AKG, CMPF, 1-carboxyethylphenylalanine, and one or more of 1-palmitoyl-2-docosahexaenoyl-GPC, 1-palmitoyl-2-dihomo-linolenoyl-GPC, and 1-stearoyl-2-docosahexaenoyl-GPC. In a further embodiment, the panel comprises AKG, CMPF, 1-carboxyethylphenylalanine, 1-palmitoyl-2-docosahexaenoyl-GPC, and one or more of 1-palmitoyl-2-dihomo-linolenoyl-GPC, and 1-stearoyl-2-docosahexaenoyl-GPC.

In an alternative embodiment, the metabolite panel comprises CMPF and one or more of AKG, 1-carboxyethylphenylalanine, 1-palmitoyl-2-docosahexaenoyl-GPC, 1-palmitoyl-2-dihomo-linolenoyl-GPC, and 1-stearoyl-2-docosahexaenoyl-GPC. In a specific embodiment, the panel comprises CMPF, AKG, and one or more 1-carboxyethylphenylalanine, 1-palmitoyl-2-docosahexaenoyl-GPC, 1-palmitoyl-2-dihomo-linolenoyl-GPC, and 1-stearoyl-2-docosahexaenoyl-GPC. In another specific embodiment, the panel comprises CMPF, AKG, 1-carboxyethylphenylalanine, and one or more of 1-palmitoyl-2-docosahexaenoyl-GPC, 1-palmitoyl-2-dihomo-linolenoyl-GPC, and 1-stearoyl-2-docosahexaenoyl-GPC. In yet another embodiment, the panel comprises CMPF, AKG, 1-carboxyethylphenylalanine, 1-palmitoyl-2-docosahexaenoyl-GPC, and one or more of 1-palmitoyl-2-dihomo-linolenoyl-GPC, and 1-stearoyl-2-docosahexaenoyl-GPC.

### BRIEF DESCRIPTION OF THE FIGURES

FIG. 1A-1B. Flowchart for selection of kidneys and metabolites analyzed. Selection of kidneys (FIG. 1A) and of metabolites analyzed (FIG. 1B).

FIG. 2. Molecular pathways, by presence in stock solution and release from kidney.

X-axis indicates the molecular super-pathway as characterized by Metabolon. The Y-axis indicates the number of molecules identified in that super-pathway. Below the pathway label is the total number of molecules in that pathway. In grey are the molecules present in the stock perfusate solution, and in green are the molecules not identified in stock samples and thus released during HMP.

FIG. 3. Perfusion time-adjusted associations of post-HMP perfusate metabolites with dcGF. The dashed horizontal line is at P=0.05, and the dashed vertical line is at HR=1. HRs are per median absolute deviation. 1-CEPA, 1-carboxyethylphenylalanine; 1-P2DL GPC, 1-palmitoyl-2-dihomo-linolenoyl-GPC (16:0/20:3n3 or 6); 1-P2DH GPC, 1-palmitoyl-2-docosahexaenoyl-GPC (16:0/22:6); 1-S2DH GPC, 1-stearoyl-2-docosahexaenoyl-GPC (18:0/22:6); AKG, alpha-ketoglutarate; CMPF, 3-carboxy-4-methyl-5-propyl-2-furanpropanoate. HRs for the 6 metabolites significantly associated with dcGF (red text; q<0.05) are presented in Table 2. re 4. dcGF-associated metabolite correlation matrix. The 3e GPCs share moderate to strong correlations. All molecules are associated with increased risk of dcGF. 1-CEP, 1-carboxyethylphenylalanine; 1P-2DH GPC, 1-palmitoyl-2-docosahexaenoyl-GPC (16:0/22:6); 1P-2DL GPC, 1-palmitoyl-2-dihomo-linolenoyl-GPC (16:0/20:3n3 or 6); 1S-2DH GPC, 1-stearoyl-2-docosahexaenoyl-GPC (18:0/22:6); AKG, alpha-ketoglutarate; CMPF, 3-carboxy-4-methyl-5-propyl-2-Furanpropanoate.

FIG. 4: Paired kidney dendrogram, from agglomerative clustering of perfusate continuous metabolites using Ward's linkage. Donor labels shown along the x-axis. “A-” prefix indicates kidney from OPO-1, “B-” prefix indicates kidney from OPO-2. Overall, 50 of 56 kidney pairs (87%) formed pairs: 38 of 39 OPO-1 pairs (97%) and 12 of 17 OPO-2 pairs (71%).

FIG. 5. Paired kidney dendrogram from agglomerative clustering of perfusate continuous metabolites using Ward's linkage. Donor labels shown along the x-axis. “A-” prefix indicates kidney from OPO-1, “B-” prefix indicates kidney from OPO-2. Overall, 50 of 56 kidney pairs (87%) formed pairs: 38 of 39 OPO-1 pairs (97%) and 12 of 17 OPO-2 pairs (71%).

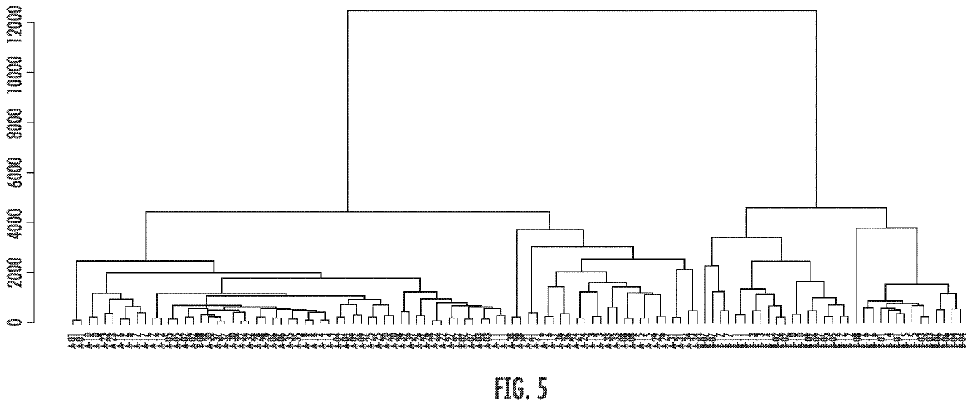

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

Physical parameters such as changes in renal resistance and perfusate flow rates are used to monitor the HMP process and infer the viability of kidneys on HMP.¹³ However, flow and resistance parameters are imperfect, non-standardized measures that vary over time, and do not appear to be linked to the biological health of the graft¹⁴ Developing an understanding of the active biological processes that occur during HMP and identifying key biomarkers directly linked to graft metabolism could improve the ability of transplant physicians to prognosticate allograft outcomes, design better perfusion solutions and strategies, and improve decision-making around the use of HMP kidneys.^{15,16} As such, there is growing interest regarding how metabolite levels in the perfusate may reflect ongoing graft metabolism and function, and prior exploratory studies have demonstrated the metabolic activity of the kidney during HMP.^{15,17}

Untargeted metabolomic profiling of biochemicals, which are 50-1000 Da in size, offers an efficient approach to exploring the active metabolic processes of kidney graft injury and repair in HMP. HMP perfusate offers an opportunity to follow the progressive changes of the allograft biological milieu as it responds to ischemic stress while remaining metabolically active. During HMP, there are changes in the composition of perfusate solution which include uptake by the kidney of nutrients, such as amino acids and glucose, to supply ongoing cell processes, release of products of metabolism and degradation, or release of cellular contents and membranes in ischemic necrosis. Interrogation of perfusate fluid allows allograft-specific biological insight beyond pump parameters and clinical characteristics such as those included in the Kidney Donor Risk Index (KDRI). Furthermore, the allograft's response to ischemic stress during HMP may provide insight to its subsequent response to additional stressors within the recipient after transplantation. Metabolic profiling would thus provide insights into the trajectory of graft function and outcomes.

Using metabolomic profiling of kidney perfusate samples collected at the beginning and end of perfusion from 2 organ procurement organizations (OPOs), the present inventor aimed to characterize metabolites that may reflect pathophysiological changes in the kidney during the duration of HMP. First, the present inventor explored the variability in the relatively novel matrix of kidney perfusate solution in both duplicate samples and paired left/right kidney samples. Second, the present inventor evaluated the association between changes in metabolites during HMP and death-censored graft failure (dcGF) and characterized differentially active metabolic processes in those kidneys that subsequently failed. Third, the present inventor performed secondary analyses on metabolites associated with dcGF for interaction with donation after cardiac death (DCD) status, association with DGF, and kidney discard.

### I. Definitions

It is understood that when combinations, subsets, groups, etc., of these metabolite biomarkers are disclosed that while specific reference of each various individual and collective combinations and permutation of these metabolites may not be explicitly disclosed, each is specifically contemplated and described herein. For example, if a particular metabolite is disclosed, each and every possible combination of that metabolite with all the other metabolites disclosed is specifically contemplated unless specifically indicated to the contrary. Thus, if a class of molecules A, B, and C are disclosed as well as a class of molecules D, E, and F and an example of a combination molecule, A-F is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application.

The term “organ” refers to a part of the body, tissue or a portion thereof. In particular embodiments, an organ comprises organs that can be transplanted or preserved ex vivo and specifically include, but are not limited to, liver, kidney, heart, lung, pancreas, small intestine and limb (e.g., arm or leg, or a portion thereof), or an extremity (e.g., hand, finger, foot, toe, or a portion thereof). In other embodiments, an organ also comprises other tissues such as tissue grafts including composite tissue allografts.

The terms “measuring” and “determining” are used interchangeably throughout, and refer to methods which include obtaining or providing a perfusion sample and/or detecting the level of a metabolite biomarker(s) in a sample. In one embodiment, the terms refer to obtaining or providing a perfusion sample and detecting the level of one or more metabolite biomarkers in the sample. In another embodiment, the terms “measuring” and “determining” mean detecting the level of one or more metabolite biomarkers in a perfusion sample. The term “measuring” is also used interchangeably throughout with the term “detecting.” In certain embodiments, the term is also used interchangeably with the term “quantitating.”

As used herein, the term “antibody” is used in reference to any immunoglobulin molecule that reacts with a specific antigen. It is intended that the term encompass any immunoglobulin (e.g., IgG, IgM, IgA, IgE, IgD, etc.) obtained from any source (e.g., humans, rodents, non-human primates, caprines, bovines, equines, ovines, etc.). Specific types/examples of antibodies include polyclonal, monoclonal, humanized, chimeric, human, or otherwise-human-suitable antibodies. “Antibodies” also includes any functional, antigen-binding fragment or derivative of any of the herein described antibodies.

As used herein, the term “antigen” is generally used in reference to any substance that is capable of reacting with an antibody. More specifically, as used herein, the term “antigen” refers to a metabolite described herein. An antigen can also refer to a synthetic peptide, polypeptide, protein or fragment of a polypeptide or protein, or other molecule which elicits an antibody response in a subject, or is recognized and bound by an antibody.

As used herein, the term “biomarker” refers to a molecule that is associated either quantitatively or qualitatively with a biological change. In certain embodiments, a “biomarker” means a compound/metabolite that is differentially present (i.e., increased or decreased) in a perfusion sample from a donor organ at one time point as compared to a perfusion sample from the donor organ at a later timepoint. A biomarker may be differentially present at any level, but is generally present at a level that is increased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more; or is generally present at a level that is decreased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent). A biomarker is preferably differentially present at a level that is statistically significant (e.g., a p-value less than 0.05 and/or a q-value of less than 0.10 as determined using, for example, either Welch's T-test or Wilcoxon's rank-sum Test). Biomarker levels can be used, in conjunction with other parameters to calculate/measure donor organ viability.

Various methodologies of the instant invention can include a step that involves comparing a value, level, feature, characteristic, property, etc. to a “suitable control,” referred to interchangeably herein as an “appropriate control,” a “control sample,” a “reference” or simply a “control.” A “suitable control,” “appropriate control,” “control sample,” “reference” or a “control” is any control or standard familiar to one of ordinary skill in the art useful for comparison purposes. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Such reference levels may also be tailored to specific techniques that are used to measure levels of biomarkers in samples (e.g., LC-MS, GC-MS, ELISA, PCR, etc.), where the levels of biomarkers may differ based on the specific technique that is used.

### II. Detection of Metabolite Biomarkers

### A. Detection by Mass Spectrometry

In one aspect, the metabolite biomarkers of the present invention may be detected by mass spectrometry, a method that employs a mass spectrometer to detect gas phase ions. Examples of mass spectrometers are time-of-flight, magnetic sector, quadrupole filter, ion trap, ion cyclotron resonance, Orbitrap, hybrids or combinations of the foregoing, and the like.

In particular embodiments, the biomarkers of the present invention are detected using selected reaction monitoring (SRM) mass spectrometry techniques. Selected reaction monitoring (SRM) is a non-scanning mass spectrometry technique, performed on triple quadrupole-like instruments and in which collision-induced dissociation is used as a means to increase selectivity. In SRM experiments two mass analyzers are used as static mass filters, to monitor a particular fragment ion of a selected precursor ion. The specific pair of mass-over-charge (m/z) values associated to the precursor and fragment ions selected is referred to as a “transition” and can be written as parent m/z→fragment m/z (e.g. 673.5→534.3). Unlike common MS based proteomics, no mass spectra are recorded in a SRM analysis. Instead, the detector acts as counting device for the ions matching the selected transition thereby returning an intensity distribution over time. Multiple SRM transitions can be measured within the same experiment on the chromatographic time scale by rapidly toggling between the different precursor/fragment pairs (sometimes called multiple reaction monitoring, MRM). Typically, the triple quadrupole instrument cycles through a series of transitions and records the signal of each transition as a function of the elution time. The method allows for additional selectivity by monitoring the chromatographic coelution of multiple transitions for a given analyte. The terms SRM/MRM are occasionally used also to describe experiments conducted in mass spectrometers other than triple quadrupoles (e.g. in trapping instruments) where upon fragmentation of a specific precursor ion a narrow mass range is scanned in MS2 mode, centered on a fragment ion specific to the precursor of interest or in general in experiments where fragmentation in the collision cell is used as a means to increase selectivity. In this application the terms SRM and MRM or also SRM/MRM can be used interchangeably since they both refer to the same mass spectrometer operating principle. As a matter of clarity, the term MRM is used throughout the text, but the term includes both SRM and MRM, as well as any analogous technique, such as e.g. highly-selective reaction monitoring, hSRM, LC-SRM or any other SRM/MRM-like or SRM/MRM-mimicking approaches performed on any type of mass spectrometer and/or, in which the peptides are fragmented using any other fragmentation method such as e.g. CAD (collision-activated dissociation (also known as CID or collision-induced dissociation), HCD (higher energy CID), ECD (electron capture dissociation), PD (photodissociation) or ETD (electron transfer dissociation).

In another specific embodiment, the mass spectrometric method comprises matrix assisted laser desorption/ionization time-of-flight (MALDI-TOF MS or MALDI-TOF). In another embodiment, method comprises MALDI-TOF tandem mass spectrometry (MALDI-TOF MS/MS). In yet another embodiment, mass spectrometry can be combined with another appropriate method(s) as may be contemplated by one of ordinary skill in the art. For example, MALDI-TOF can be utilized with trypsin digestion and tandem mass spectrometry as described herein.

In an alternative embodiment, the mass spectrometric technique comprises surface enhanced laser desorption and ionization or “SELDI,” as described, for example, in U.S. Pat. Nos. 6,225,047 and 5,719,060. Briefly, SELDI refers to a method of desorption/ionization gas phase ion spectrometry (e.g. mass spectrometry) in which an analyte (here, one or more of the biomarkers) is captured on the surface of a SELDI mass spectrometry probe. There are several versions of SELDI that may be utilized including, but not limited to, Affinity Capture Mass Spectrometry (also called Surface-Enhanced Affinity Capture (SEAC)), and Surface-Enhanced Neat Desorption (SEND) which involves the use of probes comprising energy absorbing molecules that are chemically bound to the probe surface (SEND probe). Another SELDI method is called Surface-Enhanced Photolabile Attachment and Release (SEPAR), which involves the use of probes having moieties attached to the surface that can covalently bind an analyte, and then release the analyte through breaking a photolabile bond in the moiety after exposure to light, e.g., to laser light (see, U.S. U.S. Pat. No. 5,719,060). SEPAR and other forms of SELDI are readily adapted to detecting a biomarker or biomarker panel, pursuant to the present invention.

In another mass spectrometry method, the biomarkers can be first captured on a chromatographic resin having chromatographic properties that bind the biomarkers. For example, one could capture the biomarkers on a cation exchange resin, such as CM Ceramic HyperD F resin, wash the resin, elute the biomarkers and detect by MALDI. Alternatively, this method could be preceded by fractionating the sample on an anion exchange resin before application to the cation exchange resin. In another alternative, one could fractionate on an anion exchange resin and detect by MALDI directly. In yet another method, one could capture the biomarkers on an immuno-chromatographic resin that comprises antibodies that bind the biomarkers, wash the resin to remove unbound material, elute the biomarkers from the resin and detect the eluted biomarkers by MALDI or by SELDI.

### B. Detection by Immunoassay

In other embodiments, the metabolite biomarkers of the present invention can be detected and/or measured by immunoassay. Immunoassay requires specific capture reagents/binding agent, such as antibodies, to capture the biomarkers. Many antibodies are available commercially. Antibodies also can be produced by methods well known in the art, e.g., by immunizing animals with the biomarkers. Biomarkers can be isolated from samples based on their binding characteristics.

The present invention contemplates traditional immunoassays including, for example, sandwich immunoassays including ELISA or fluorescence-based immunoassays, immunoblots, Western Blots (WB), as well as other enzyme immunoassays. Nephelometry is an assay performed in liquid phase, in which antibodies are in solution. Binding of the antigen to the antibody results in changes in absorbance, which is measured. In a SELDI-based immunoassay, a biospecific capture reagent for the biomarker is attached to the surface of an MS probe, such as a pre-activated protein chip array. The biomarker is then specifically captured on the biochip through this reagent, and the captured biomarker is detected by mass spectrometry.

In certain embodiments, the levels of the metabolite biomarkers employed herein are quantified by immunoassay, such as enzyme-linked immunoassay (ELISA) technology. In specific embodiments, the levels of expression of the biomarkers are determined by contacting the biological sample with antibodies, or antigen binding fragments thereof, that selectively bind to the metabolite biomarkers; and detecting binding of the antibodies, or antigen binding fragments thereof, to the metabolite biomarkers. In certain embodiments, the binding agents employed in the disclosed methods and compositions are labeled with a detectable moiety.

For example, the level of a metabolite biomarker in a sample can be assayed by contacting the biological sample with an antibody, or antigen binding fragment thereof, that selectively binds to the target biomarker (referred to as a capture molecule or antibody or a binding agent), and detecting the binding of the antibody, or antigen-binding fragment thereof, to the biomarker. The detection can be performed using a second antibody to bind to the capture antibody complexed with its target metabolite biomarker. Kits for the detection of biomarkers as described herein can include pre-coated strip plates, biotinylated secondary antibody, standards, controls, buffers, streptavidin-horse radish peroxidase (HRP), tetramethyl benzidine (TMB), stop reagents, and detailed instructions for carrying out the tests including performing standards.

In a further aspect, the present disclosure provides compositions that can be employed in the disclosed methods. In certain embodiments, such compositions a solid substrate and a plurality of binding agents immobilized on the substrate, wherein each of the binding agents is immobilized at a different, indexable, location on the substrate and the binding agents selectively bind to a plurality of metabolite biomarkers disclosed herein. In a specific embodiment, the locations are pre-determined. In other embodiments, kits are provided that comprise such compositions. In certain embodiments, the plurality of metabolite biomarkers includes one or more of the metabolites described herein including alpha-ketoglutarate (AKG), 3-carboxy-4-methyl-5-propyl-2-furanpropanoate (CMPF), 1-carboxyethylphenylalanine, 1-palmitoyl-2-docosahexaenoyl-glycerophosphocholine (GPC), 1-palmitoyl-2-dihomo-linolenoyl-GPC, and 1-stearoyl-2-docosahexaenoyl-GPC.

In other embodiments, such compositions additionally comprise binding agents that selectively bind to other biomarkers. Binding agents that can be employed in such compositions include, but are not limited to, antibodies, or antigen-binding fragments thereof, aptamers, lectins, other metabolites and the like.

Although antibodies are useful because of their extensive characterization, any other suitable agent (e.g., a peptide, an aptamer, or a small organic molecule) that specifically binds a metabolite biomarker of the present invention is optionally used in place of the antibody in the above described immunoassays. For example, an aptamer that specifically binds a metabolite biomarker and/or one or more of its further breakdown products might be used. Aptamers are nucleic acid-based molecules that bind specific ligands. Methods for making aptamers with a particular binding specificity are known as detailed in U.S. Pat. No. 5,475,096; 5,670,637; 5,696,249; 5,270, 163; 5,707,796; 5,595,877; 5,660,985; 5,567,588; 5,683,867; 5,637,459; and 6,011,020.

In specific embodiments, the assay performed on the biological sample can comprise contacting the biological sample with one or more capture agents (e.g., antibodies, peptides, aptamer, etc., combinations thereof) to form a metabolite biomarker: capture agent complex. The complexes can then be detected and/or quantified.

In one method, a first, or capture, binding agent, such as an antibody that specifically binds the metabolite biomarker of interest, is immobilized on a suitable solid phase substrate or carrier. The test biological sample is then contacted with the capture antibody and incubated for a desired period of time. After washing to remove unbound material, a second, detection, antibody that binds to a different, non-overlapping, epitope on the biomarker is then used to detect binding of the metabolite biomarker to the capture antibody. The detection antibody is preferably conjugated, either directly or indirectly, to a detectable moiety. Examples of detectable moieties that can be employed in such methods include, but are not limited to, cheminescent and luminescent agents; fluorophores such as fluorescein, rhodamine and eosin; radioisotopes; colorimetric agents; and enzyme-substrate labels, such as biotin.

Solid phase substrates, or carriers, that can be effectively employed in such assays are well known to those of skill in the art and include, for example, 96 well microtiter plates, glass, paper, chips and microporous membranes constructed, for example, of nitrocellulose, nylon, polyvinylidene difluoride, polyester, cellulose acetate, mixed cellulose esters and polycarbonate. Suitable microporous membranes include, for example, those described in US Patent Application Publication no. US 2010/0093557 A1. Methods for the automation of immunoassays are well known in the art and include, for example, those described in U.S. Pat. Nos. 5,885,530, 4,981,785, 6,159,750 and 5,358,691.

The presence of several different metabolite biomarkers in a test sample can be detected simultaneously using a multiplex assay, such as a multiplex ELISA. Multiplex assays offer the advantages of high throughput, a small volume of sample being required, and the ability to detect different proteins across a board dynamic range of concentrations.

In certain embodiments, such methods employ an array, wherein multiple binding agents (for example capture antibodies) specific for multiple biomarkers are immobilized on a substrate, such as a membrane, with each capture agent being positioned at a specific, pre-determined, location on the substrate. Methods for performing assays employing such arrays include those described, for example, in US Patent Application Publication nos. US2010/0093557A1 and US2010/0190656A1, the disclosures of which are hereby specifically incorporated by reference.

Multiplex arrays in several different formats based on the utilization of, for example, flow cytometry, chemiluminescence or electron-chemiluminesence technology, are well known in the art. Flow cytometric multiplex arrays, also known as bead-based multiplex arrays, include the Cytometric Bead Array (CBA) system from BD Biosciences (Bedford, Mass.) and multi-analyte profiling (xMAP®) technology from Luminex Corp. (Austin, Tex.), both of which employ bead sets which are distinguishable by flow cytometry. Each bead set is coated with a specific capture antibody. Fluorescence or streptavidin-labeled detection antibodies bind to specific capture antibody-biomarker complexes formed on the bead set. Multiple biomarkers can be recognized and measured by differences in the bead sets, with chromogenic or fluorogenic emissions being detected using flow cytometric analysis. In an alternative format, a multiplex ELISA from Quansys Biosciences (Logan, Utah) coats multiple specific capture antibodies at multiple spots (one antibody at one spot) in the same well on a 96-well microtiter plate. Chemiluminescence technology is then used to detect multiple biomarkers at the corresponding spots on the plate.

### C. Other Methods for Detecting Metabolite Biomarkers

In several embodiments, the metabolite biomarkers of the present invention may be detected by means of an electrochemicaluminescent assay, for example, developed by Meso Scale Discovery (Gaithersburg, MD). Electrochemiluminescence detection uses labels that emit light when electrochemically stimulated. Background signals are minimal because the stimulation mechanism (electricity) is decoupled from the signal (light). Labels are stable, non-radioactive and offer a choice of convenient coupling chemistries. They emit light at ~620 nm, eliminating problems with color quenching. See U.S. Pat. Nos. 7,497,997; 7,491,540; 7,288,410; 7,036,946; 7,052,861; 6,977,722; 6,919,173; 6,673,533; 6,413,783; 6,362,011; 6,319,670; 6,207,369; 6,140,045; 6,090,545; and 5,866,434. See also U.S. Patent Applications Publication No. 2009/0170121; No. 2009/006339; No. 2009/0065357; No. 2006/0172340; No. 2006/0019319; No. 2005/0142033; No. 2005/0052646; No. 2004/0022677; No. 2003/0124572; No. 2003/0113713; No. 2003/0003460; No. 2002/0137234; No. 2002/0086335; and No. 2001/0021534.

The metabolite biomarkers of the present invention can also be detected by other suitable methods. Detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry). Furthermore, a sample may also be analyzed by means of a chip. Chips generally comprise solid substrates and have a generally planar surface, to which a capture reagent (also called an adsorbent or affinity reagent) is attached. Frequently, the surface of a chip comprises a plurality of addressable locations, each of which has the capture reagent bound there. These include, for example, chips produced by Advion, Inc. (Ithaca, NY).

### III. Determination of a Donor Organ's Status

### A. Metabolite Biomarker Panels

The present invention relates to the use of metabolite biomarkers to determine viability of a donor organ. In particular embodiments, the biomarkers of the present invention include, but are not limited to, alpha-ketoglutarate (AKG), 3-carboxy-4-methyl-5-propyl-2-furanpropanoate (CMPF), 1-carboxyethylphenylalanine, 1-palmitoyl-2-docosahexaenoyl-glycerophosphocholine (GPC), 1-palmitoyl-2-dihomo-linolenoyl-GPC, and 1-stearoyl-2-docosahexaenoyl-GPC.

The power of a diagnostic test to correctly predict status is commonly measured as the sensitivity of the assay, the specificity of the assay or the area under a receiver operated characteristic (“ROC”) curve. Sensitivity is the percentage of true positives that are predicted by a test to be positive, while specificity is the percentage of true negatives that are predicted by a test to be negative. An ROC curve provides the sensitivity of a test as a function of 1-specificity. The greater the area under the ROC curve, the more powerful the predictive value of the test. Other useful measures of the utility of a test are positive predictive value and negative predictive value. Positive predictive value is the percentage of people who test positive that are actually positive. Negative predictive value is the percentage of people who test negative that are actually negative.

In particular embodiments, the metabolite biomarker panels of the present invention may show a statistical difference in different organ viability statuses of at least p<0.05, p<10⁻², p<10⁻³, p<10⁻⁴ or p<10⁻⁵. Diagnostic tests that use these biomarkers may show an ROC of at least 0.6, at least about 0.7, at least about 0.8, or at least about 0.9.

Furthermore, in certain embodiments, the values measured for markers of a metabolite biomarker panel are mathematically combined and the combined value is correlated to the underlying diagnostic question. Biomarker values may be combined by any appropriate state of the art mathematical method. Well-known mathematical methods for correlating a marker combination to a disease status employ methods like discriminant analysis (DA) (e.g., linear-, quadratic-, regularized-DA), Discriminant Functional Analysis (DFA), Kernel Methods (e.g., SVM), Multidimensional Scaling (MDS), Nonparametric Methods (e.g., k-Nearest-Neighbor Classifiers), PLS (Partial Least Squares), Tree-Based Methods (e.g., Logic Regression, CART, Random Forest Methods, Boosting/Bagging Methods), Generalized Linear Models (e.g., Logistic Regression), Principal Components based Methods (e.g., SIMCA), Generalized Additive Models, Fuzzy Logic based Methods, Neural Networks and Genetic Algorithms based Methods. The skilled artisan will have no problem in selecting an appropriate method to evaluate a biomarker combination of the present invention. In one embodiment, the method used in a correlating a biomarker combination of the present invention, e.g. to determine/calculate donor organ viability, is selected from DA (e.g., Linear-, Quadratic-, Regularized Discriminant Analysis), DFA, Kernel Methods (e.g., SVM), MDS, Nonparametric Methods (e.g., k-Nearest-Neighbor Classifiers), PLS (Partial Least Squares), Tree-Based Methods (e.g., Logic Regression, CART, Random Forest Methods, Boosting Methods), or Generalized Linear Models (e.g., Logistic Regression), and Principal Components Analysis. Details relating to these statistical methods are found in the following references: Ruczinski et al., 12 J. OF COMPUTATIONAL AND GRAPHICAL STATISTICS 475-511 (2003); Friedman, J. H., 84 J. OF THE AMERICAN STATISTICAL ASSOCIATION 165-75 (1989); Hastie, Trevor, Tibshirani, Robert, Friedman, Jerome, The Elements of Statistical Learning, Springer Series in Statistics (2001); Breiman, L., Friedman, J. H., Olshen, R. A., Stone, C. J. Classification and regression trees, California: Wadsworth (1984); Breiman, L., 45 MACHINE LEARNING 5-32 (2001); Pepe, M. S., The Statistical Evaluation of Medical Tests for Classification and Prediction, Oxford Statistical Science Series, 28 (2003); and Duda, R. O., Hart, P. E., Stork, D. G., Pattern Classification, Wiley Interscience, 2nd Edition (2001).

The Examples below further illustrate additional embodiments for determining viability of a donor organ.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Materials and Methods

Study population. This is an ancillary study to the Deceased Donor Study (DDS), an ongoing multicenter, observational, cohort study of deceased donors and their kidney recipients enrolled between 2010 and 2013. The DDS study population and methods have been described in detail elsewhere^{14,18-20}. The investigator team worked closely with 2 OPOs (New York Organ Donor Network [OPO-1], New York, NY; and Gift of Life Michigan [OPO-2], Ann Arbor, MI) that collected perfusate samples.

The cohort was comprised of transplanted kidneys that underwent HMP, from donors at least 16 years of age whose surrogates provided consent for research. Deceased donors were included if at least 1 kidney underwent HMP. Kidneys were excluded if no perfusate samples were obtained. OPO personnel followed institutional protocols for managing donors. The study was approved by OPO scientific review committees and IRBs for the investigators.

Perfusate collection and measurement. All kidneys were individually pumped using the LifePort Kidney Transporter (Organ Recovery Systems, Itasca, IL). OPO personnel managed the perfusion machines according to the OPO's protocol. Perfusate samples were collected from the perfusion machine at 2 timepoints: 1 sample within 10 minutes of starting perfusion, referred to as the baseline sample, and a second sample just before the OPO transferred management of the kidney to the recipient center, referred to as the post-HMP sample. The timing of sample collections was recorded by OPO personnel. Each sample was transported on ice and stored at −80° C. at the OPO until monthly batch shipments to the coordinating center. Samples were subsequently processed at the coordinating center following a single controlled thaw, separated into bar-coded aliquots, and stored at −80° C. without the addition of protease inhibitors until metabolite measurement.

Perfusate measurements. All samples were measured by Metabolon Inc. (Durham, NC) using high-performance liquid chromatography/tandem accurate mass spectrometry methods, as described previously.²¹ All analysis and preprocessing of spectral peaks to quantify biochemicals in samples were performed by Metabolon. Four samples of perfusate solution, referred to as stock samples, were measured alongside the kidney perfusate samples collected during HMP. Sample quality control was performed based on the consistency of baseline and post-HMP perfusate time labels and samples for which the labeled collection time of baseline perfusate was missing or invalid were excluded. In addition, 42 pairs of duplicate samples were measured to quantify coefficient of variation (% CVs) for each metabolite. Additional measurement details provided in Supplemental Materials and Methods below.

Data sources. To ascertain donor and recipient characteristics and outcomes, study databases were linked to the United Network for Organ Sharing (UNOS) database. UNOS is contracted to supply data to the Organ Procurement and Transplantation Network (OPTN). The OPTN data system includes data on all donor, wait-listed candidates, and transplant recipients in the United States, submitted by the members of the OPTN, and has been described elsewhere. The Health Resources and Services Administration, U.S. Department of Health and Human Services provides oversight to the activities of the OPTN contractor. Through review of OPO charts, donor data was augmented with additional elements, including admission serum creatinine, and perfusion time. Chart review was performed on recipients at participant transplant centers in the present inventor's study network who received kidneys from enrolled donors. Trained site coordinators reviewed medical records and recorded detailed recipient characteristics and outcomes.

Outcome definitions. The primary outcome was dcGF, defined as return to dialysis after transplantation during the study follow-up. The loss of the kidney graft due to recipient death was censored in this analysis. In a secondary analysis, the outcome of DGF was also used, defined by the need for >1 dialysis session in the first week or a serum creatinine reduction ratio <25% within the first 48 hours post-transplantation.²²

Statistics. Descriptive statistics were reported as mean (SD) or median (IQR) for continuous variables and as frequency (percentage) for categorical variables. The KDRI was calculated based on the following donor characteristics: age; sex; race; height; weight; history of hypertension; history of diabetes; hepatitis C serostatus; stroke as the cause of death; donation after cardiac determination of death status; and terminal serum creatinine²³. The 2010 kidney donor profile index (KDPI) was calculated from the KDRI, as per convention²³.

For metabolites with >50% of samples above the minimum detected level in post-HMP perfusate measurements, biochemical measurements were scaled such that 1 unit equals 1 median absolute deviation (MAD). In evaluating outcome associations, metabolites with ≤50% samples above the minimum detected level (105 of 388 metabolites) were dichotomized. Non-dichotomized metabolites are referred to as continuous metabolites.

To estimate the dcGF HR per MAD, the present inventor fit Cox proportional hazard models adjusted for perfusion time, and then additionally for KDPI. The cluster effect of paired kidneys from the same donor was accounted for using robust sandwich estimates by donor. De novo metabolites were considered significantly dcGF-associated after false-discovery correction. Additional details on analyses found in the Supplemental Materials and Methods.

Study approval. The study was approved by the institutional review boards of all participating institutions, and written informed consent was obtained from all participants or their surrogates.

### Results

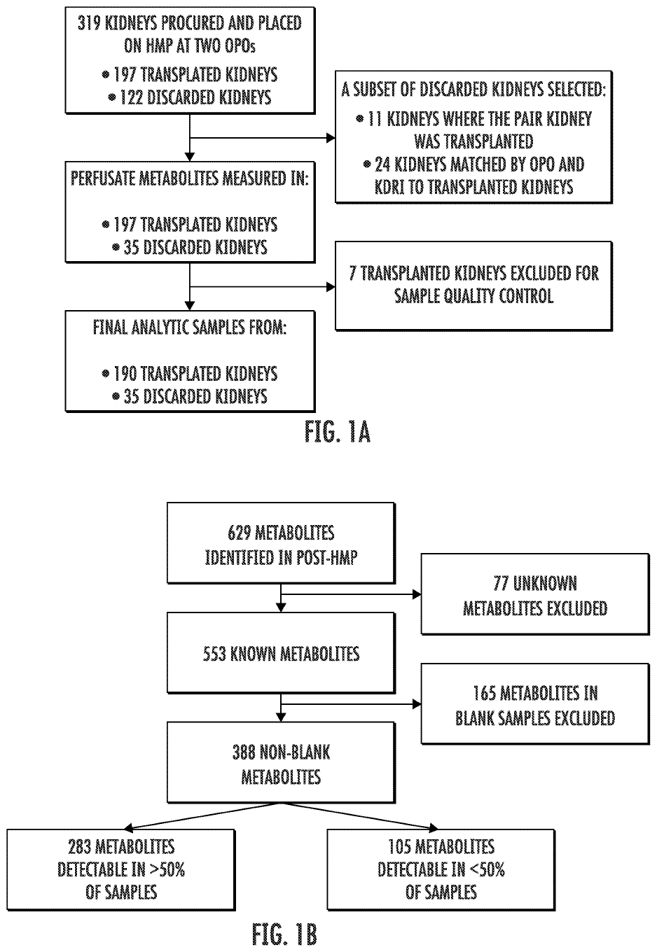

Donor and recipient characteristics. Perfusate samples from all 197 kidneys that underwent HMP and were transplanted were selected for untargeted metabolomic analysis (FIG. 1A). Perfusate from 35 discarded kidneys matched by OPO and KDRI were also analyzed to compare discarded vs dcGF and non-dcGF kidneys. After sample quality control, 7 samples from transplanted kidneys were excluded, and perfusate from 190 individually transplanted kidneys and 35 discarded kidneys that underwent HMP were included.

The transplanted kidneys in the study were contributed by 147 deceased donors, whose characteristics are provided in Table 1. Mean KDRI was 1.42±0.43 and mean admission-to-procurement time was 6.6±5 days. Individual kidneys underwent 9.9±5.7 hours of HMP before transplantation. The recipient cohort for the 190 HMP kidneys had a mean age of 57±14 years, 65% were male, 45% were black, and mean duration on dialysis was 58±37 months (Table 1). The donor characteristics of 35 discarded kidneys are presented in Table 4.

Perfusate metabolomics measurements and quality. Metabolomics analysis quantified 629 unique metabolites from perfusate samples, 76 of which were unknown chemicals and were excluded from the analysis (FIG. 1B). Of the 553 known metabolites, 165 were identified in the stock perfusate solution samples, and are referred to as “stock” metabolites. The other 388 metabolites were referred as “de novo” metabolites which appeared in perfusate while the graft was being perfused (Appendix, available online (Liu et al., 103 KIDNEY INTERNATIONAL 762-771 (2023), “Untargeted Metabolomics of Perfusate and Their Association with Hypothermic Machine Perfusion and Allograft Failure”), which is specifically incorporated by reference herein).

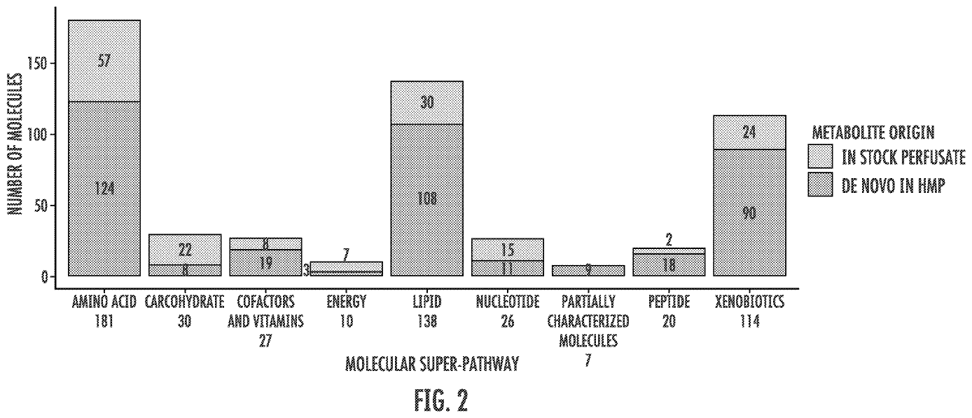

The set of metabolites detected in samples from each of the 2 OPOs were consistent, with 547 (99%) metabolites detected among samples from OPO-1 and 537 (97%) detected among samples from OPO-2 demonstrating consistency in the biological processes across sites. The 553 known metabolites included 181 amino acids, 138 lipids, 114 xenobiotics, 30 carbohydrates, 27 cofactors/vitamins, 26 nucleotides, 20 peptides, 10 energy molecules, and 7 partially characterized molecules (FIG. 2). Quantified molecules ranged from 74 to 835 Da in size. The median % CV of continuous de novo and stock metabolites was 19.0 (IQR: 15.4-23.4) and 16.5 (IQR: 11.6-22.6), respectively (Appendix). Of known metabolites identified in perfusate, 13% (74) demonstrated significant associations between concentration changes during perfusion and perfusion time (P<0.05). Of these changes, 90% (67) were increases in concentration over time with continued perfusion (Appendix).

The similarity of paired donor kidneys versus non-paired kidneys was compared to explore the consistency of the perfusate metabolome through correlation and clustering. Among the 112 kidney pairs from 56 donors, the post-HMP perfusate metabolites were strongly correlated, with overall median rₛ=0.88 (IQR: 0.82-0.91), while non-paired kidneys were weakly correlated, with median rₛ=0.13 (IQR: −0.01 to 0.26). After adjustment for paired perfusion time differences, the metabolites remained strongly correlated, with median R2=0.782 with an IQR of [0.590, 0.890]. Among the metabolites, the median Pearson correlation between paired left and right kidneys was 0.84 (IQR: 0.73-0.92), altogether suggesting a biological consistency of the perfusate metabolome between left and right kidneys (Appendix).

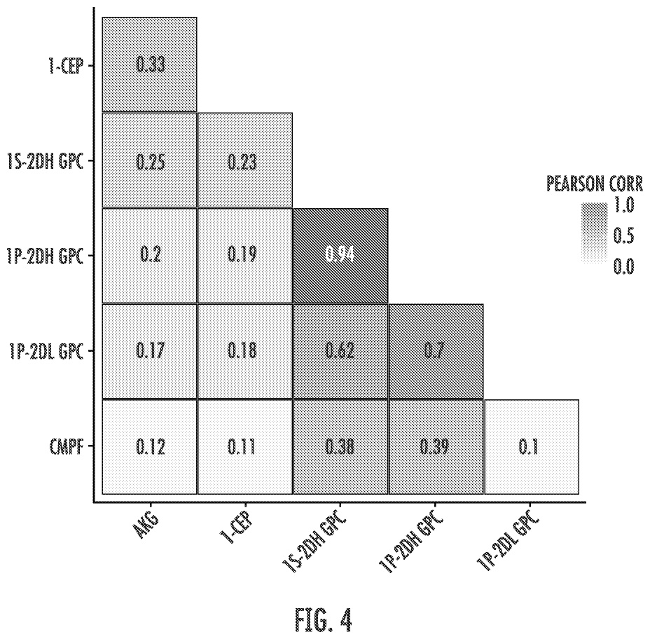

To explore the structure and specificity of the allograft perfusate metabolomes, unsupervised agglomerative clustering was performed. Overall, 50 of 56 kidney pairs (87%) formed unsupervised pairs (FIG. 4). The post-HMP perfusate metabolome of paired kidneys was not only strongly correlated, but also distinguishable from other donor kidneys, suggesting biological consistency.

Association of post-HMP perfusate metabolites with dcGF in recipients. Of the 190 recipients, 33 (17%) experienced dcGF over a median follow-up of 5.0 (IQR: 3.0-6.1) years. The event rate for dcGF was 38.1 (95% CI: 26.3-56.6) per 1000 person-years.

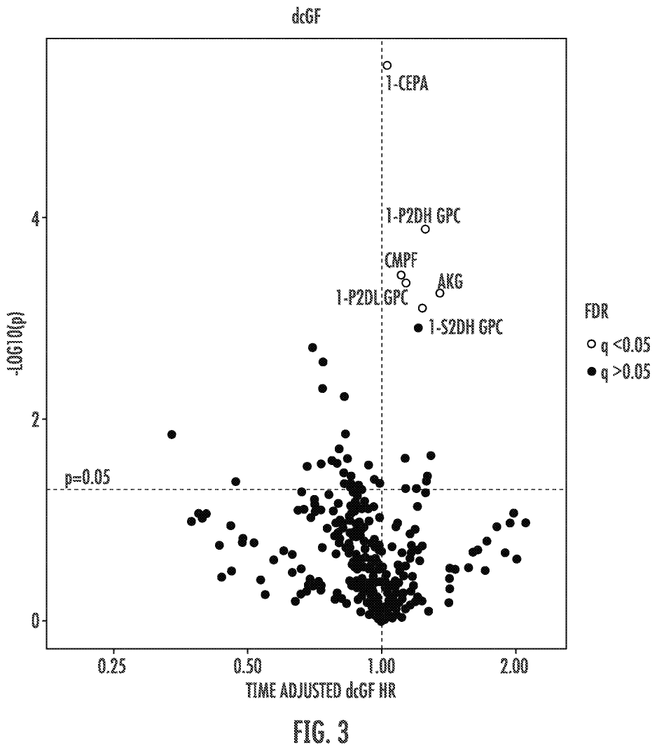

After adjusting for duration of HMP, significant associations in post-HMP perfusate metabolites were observed with dcGF for 6 metabolites with false discovery rate-corrected P<0.05 (q<0.05) (Table 2) and for 42 metabolites with P<0.05 (FIG. 3, Appendix). These metabolites include alpha-ketoglutarate (AKG), 3-carboxy-4-methyl-5-propyl-2-furanpropanoate (CMPF), 1-carboxyethylphenylalanine, 1-palmitoyl-2-docosahexaenoyl-glycerophosphocholine (GPC) (16:0/22:6), 1-palmitoyl-2-dihomo-linolenoyl-GPC (16:0/20:3n3 or 6), and 1-stearoyl-2-docosahexaenoyl-GPC (18:0/22:6). No significant interactions with OPO (P<0.1) were found for these metabolites. The strongest association was seen with AKG, which had a 45% increased risk for dcGF per each median absolute deviation (MAD) increase. While no metabolites were associated with reduced risk of dcGF after false discovery correction, 26 of the 42 metabolites with dcGF association p<0.05 were associated with reduced risk (Appendix).

Among the 6 dcGF-associated metabolites, the 3 GPCs were strongly correlated with each other (FIG. 4), and CMPF was moderately correlated with 1-palmitoyl-2-docosahexaenoyl-GPC and 1-stearoyl-2-docosahexaenoyl-GPC (18:0/22:6). AKG and 1-carboxyethylphenylalanine were only weakly correlated with other dcGF-associated metabolites.

Stratification of dcGF-association by donation after circulatory death and high-KDPI status. Whether high-risk donors demonstrated different dcGF associations for the 6 dcGF-associated metabolites was also evaluated, stratifying by both donation after circulatory death (DCD, n=39) status and high-KDPI (KDPI ≥80, n=47). In testing for DCD interactions in dcGF associations, significant interactions (P<0.05) were observed in 4 metabolites, including AKG, 1-carboxyethylphenylalanine, 1-palmitoyl-2-dihomo-linolenoyl-GPC, and 1-stearoyl-2-docosahexaenoyl-GPC (Table 5). In all 4 of these interactions, risk associations were notably greater in the DCD group, with HRs ranging up to 1.69 (alpha-ketoglutarate). Notably, though the overall HR of 1-carboxyethylphenylalanine was 1.04, the HR in DCD donors was 1.46. While hazard ratios were attenuated in these 4 metabolites in non-DCD donors, metabolites were still significantly associated with dcGF in the non-DCD group for all metabolites except for 1-palmitoyl-2-dihomo-linolenoyl-GPC (16:0/20:3n3 or 6). No significant interactions were found with high-KDPI.

dcGF-associated pathways in HMP. To profile active cellular pathways differentially activated in dcGF kidneys, the 42 P<0.05 dcGF-associated perfusate metabolites were analyzed using Ingenuity Pathway Analysis. Of the 388 de novo metabolites, 308 were linked in the Ingenuity database. Among the 42 metabolites used, 40 were linked to the database. The top functional pathways enriched in dcGF were cell death, accumulation of fatty acids, amino acid transport, production of reactive oxygen species (ROS), and metabolism of glutathione (Table 3). Metabolite ratios in these pathways suggested increases in ROS production and fatty acid accumulation in dcGF kidneys with high statistical confidence.

Post-HMP perfusate dcGF associations after adjusting for baseline. To determine whether the dcGF associations for post-HMP perfusate metabolites were associated with production during perfusion, as opposed to their initial concentration in perfusate measured at base, a supplementary analysis was performed in which models for the 6 dcGF-associated metabolites were adjusted for baseline perfusate levels and perfusion time (Table 6). Similar strengths of association but wider confidence intervals for all biomarkers were observed, overall suggesting the increases in metabolite levels throughout perfusion were significantly associated with dcGF. One metabolite, 1-carboxyethylphenylalanine, lost significance in this analysis.

Associations of post-HMP perfusate metabolites with delayed graft function. The occurrence of DGF as a short-term secondary outcome was also explored. Of the 190 kidney recipients in this study, 120 (63%) experienced DGF. Four of the six dcGF-associated metabolites were associated with DGF, including CMPF, AKG, 1-stearoyl-2-docosahexaenoyl-GPC (18:0/22:6), and 1-palmitoyl-2-docosahexaenoyl-GPC (16:0/22:6) (Table 7).

Comparison of metabolite levels with discarded kidneys. Perfusate measurements from the 35 discarded kidneys were compared against perfusate measurements from kidneys that did or did not experience dcGF. The 35 kidneys were on HMP for a mean of 10.9±5.6 hours before being discarded. No differences in the post-HMP or baseline concentrations of the 6 dcGF-associated metabolites were observed between discarded kidneys and all transplanted kidneys, kidneys with dcGF, and kidneys with no dcGF (Table 8). These findings suggest no differences in the metabolic functioning of the kidneys at the time of discard.

### Discussion

In this study, an untargeted metabolomic profiling of 190 kidneys from 2 OPOs was performed to identify 553 metabolites in allograft HMP perfusate. The present inventor investigated the association between 388 de novo metabolites and dcGF in 190 kidneys with median follow-up of 5 years to identify 6 dcGF-associated metabolites: AKG, CMPF, 1-carboxyethylphenylalanine, and 3 GPCs. Given their significant interaction and greater risk association with DCD, these metabolites may be especially helpful in assessing viability of kidneys undergoing HMP during the allocation process. The association of these metabolites with dcGF after adjusting for their measurements at baseline, perfusion time, and KDPI suggests that, specifically, their de novo production from the kidney during HMP is associated with early graft failure. The present inventor characterized actively changing molecules during HMP by identifying 74 de novo metabolites significantly changing over time, demonstrating that perfused kidneys are biologically active. Additionally, the present inventor demonstrated that the dcGF-associated metabolites did not differ significantly between discarded and transplanted kidneys; these similarities in dcGF-associated metabolites may further call into question current decision-making practices for discarding kidneys.^{24,25} Using biological markers may improve the accuracy of the discard process and help further expand the deceased-donor pool for transplantable organs.

The present inventor also rigorously characterized the variation and consistency of the perfusate matrix for metabolomics in this platform by quantifying the % CV of the 553 metabolites identified. The present inventor demonstrated allograft perfusate metabolomes were experimentally consistent through blind duplicate pairing, and biologically consistent through the unsupervised pairing of left and right paired kidneys from the same donor. Altogether, the present inventor demonstrated that is it feasible to obtain high quality measurements from perfusate cover the duration of perfusion.

In addition to preserving the graft, HMP provides a potential window for prognostic and therapeutic assessment, which is increasingly important as deceased-donor transplantation continues to expand with higher-risk kidneys. While 20% of all kidneys are pumped, and an even greater fraction of higher-risk kidneys, upwards of 30% of kidneys are discarded afterwards. After validation, these 6 dcGF-associated metabolites could help make better organ offer decisions by contributing to an objective evaluation of allograft offers, thereby reducing the discard of potentially viable organs.

Pathway analysis of the post-HMP perfusate of kidneys that went on to develop dcGF revealed increases in ROS production and in the concentration of fatty acids compared with kidneys that did not develop dcGF. It has been well-established that ROS production during ischemia-reperfusion injury is a major mechanism of injury during transplantation.^{26,27} Other studies have found that high-density lipoprotein efflux from macrophage foam cells predicts kidney graft failure.²⁸ Hyperlipidemia has also been established as a risk factor for chronic allograft dysfunction.²⁹ Pathway analysis also indicated that pathways in cell death, glutathione metabolism, and amino acid transport were also altered. HMP perfusate represents a promising matrix in which to study mechanisms implicated in allograft health, especially processes that are perturbed or activated during transport.

AKG is an important citric acid cycle intermediate and can be generated from various sources, including directly from glutamine and indirectly from glucose and fatty acids^{15,30} Infusion of AKG post-coronary operations may have protective effects by increasing renal blood flow³¹. There is also evidence that infusion of AKG reduces proximal tubule injury³². Higher levels of AKG in the perfusate may suggest a greater response from the kidney to oxidative stress and injury. In this way, AKG may be a marker of greater injury and worse graft outcomes.

Three of the 6 metabolites associated with worse graft survival are GPCs. GPCs are important components of cell membranes, and elevated extracellular concentration of GPCs may be indicative of cellular necrosis resulting from ischemic damage.^{33,34} GPCs also specifically protect renal medullary cells from high extracellular osmolarity, and are concentrated as extracellular NaCl and urea increase.³⁵ Thus far however, medullary damage has not been strongly linked to cortical rejection.³⁶ Metabolically, a number of GPCs can preserve mitochondrial complex I respiratory function and thereby reduce the effects of ROS generated from ischemic stress; thus the loss of intracellular and membrane GPCs could reduce the allograft's capacity to respond to subsequent post-transplant reperfusion stress.³⁷ Altogether, the association between higher levels of perfusate GPCs and poorer graft survival may suggest that GPC levels are markers of increased response to ischemic injury.

CMPF may also be indicative of permanent or underlying kidney injury that could lead to worse graft survival. CMPF is a protein-bound uremic toxin that is significantly increased in chronic kidney disease and induces proximal tubular cell damage via the generation of radical intermediates.^{38,39} CMPF can also inhibit mitochondrial respiration.⁴⁰ Lastly, increased levels of 1-carboxyethylphenylalanine, a phenylalanine derivate, may also be a sign of chronic kidney injury. The conversion of phenylalanine to tyrosine is reduced in settings of chronic kidney injury.⁴¹⁻⁴³

In this large multicenter cohort, 6 de novo metabolites were associated with dcGF. The associations were stronger in DCD kidneys, where evaluation of graft quality is challenging and discard rates remain high. Through a better understanding of differentially active pathways in dcGF, transplant researchers and clinicians may be able to develop validated biological markers that reflect allograft physiology to aid in prognostication and post-transplant management.

### REFERENCES

1. Peng P, Ding Z, He Y, Zhang J, Wang X, Yang Z. Hypothermic Machine Perfusion Versus Static Cold Storage in Deceased Donor Kidney Transplantation: A Systematic Review and Meta-Analysis of Randomized Controlled Trials. Artif Organs. 2019; 43(5):478-489. doi: 10.1111/aor.13364.
2. Ravaioli M, De Pace V, Angeletti A, et al. Hypothermic Oxygenated New Machine Perfusion System in Liver and Kidney Transplantation of Extended Criteria Donors: First Italian Clinical Trial. Scientific Reports. 2020; 10(1):6063. doi: 10.1038/s41598-020-62979-9.
3. Tingle S J, Figueiredo R S, Moir J A, et al. Hypothermic machine perfusion is superior to static cold storage in deceased donor kidney transplantation: A meta-analysis. Clinical Transplantation. 2020; 34(4):e13814. doi: https://doi.org/10.1111/ctr.13814.
4. Fu Z, Ye Q, Zhang Y, et al. Hypothermic Machine Perfusion Reduced Inflammatory Reaction by Downregulating the Expression of Matrix Metalloproteinase 9 in a Reperfusion Model of Donation After Cardiac Death. Artificial Organs. 2016; 40(6):E102-E111. doi: 10.1111/aor.12658.
5. Zhang Y, Fu Z, Zhong Z, et al. Hypothermic Machine Perfusion Decreases Renal Cell Apoptosis During Ischemia/Reperfusion Injury via the Ezrin/AKT Pathway. Artificial Organs. 2016; 40(2):129-135. doi: 10.1111/aor.12534.
6. Guzzi F, Knight S R, Ploeg R J, Hunter J P. A systematic review to identify whether perfusate biomarkers produced during hypothermic machine perfusion can predict graft outcomes in kidney transplantation. Transplant International. 2020; 33(6):590-602. doi: 10.1111/tri. 13593.
7. Cooper M, Formica R, Friedewald J, et al. Report of National Kidney Foundation Consensus Conference to Decrease Kidney Discards. Clinical Transplantation. 2019; 33(1):e13419. doi: 10.1111/ctr.13419.
8. Chang A, Schaubel D E, Chen M, Abt P L, Bittermann T. Trends and Outcomes of Hypothermic Machine Perfusion Preservation of Kidney Allografts in Simultaneous Liver and Kidney Transplantation in the United States. Transpl Int. 2022; 0. doi: 10.3389/ti.2022.10345.

9 Adler J T, Husain S A, King K L, Mohan S. Greater complexity and monitoring of the new Kidney Allocation System: Implications and unintended consequences of concentric circle kidney allocation on network complexity. Am J Transplant. 2021; 21(6):2007-2013. doi: 10.1111/ajt.16441.
10. Rohan V S, Pilch N, McGillicuddy J, et al. Early Assessment of National Kidney Allocation Policy Change. Journal of the American College of Surgeons. 2022; 234(4):565-570. doi: 10.1097/XCS.0000000000000096.
11. Sharma N, Mahajan A, Qazi Y A. Marginal kidney transplantation: the road less traveled. Current Opinion in Organ Transplantation. 2019; 24(1):92-96. doi: 10.1097/MOT.0000000000000603.
12. Woodside K J, Merion R M, Leichtman A B, et al. Utilization of Kidneys With Similar Kidney Donor Risk Index Values From Standard Versus Expanded Criteria Donors. American Journal of Transplantation. 2012; 12(8):2106-2114. doi: 10.1111/j.1600-6143.2012.04146.
13. Jochmans I, Moers C, Smits J M, et al. The Prognostic Value of Renal Resistance During Hypothermic Machine Perfusion of Deceased Donor Kidneys. American Journal of Transplantation. 2011; 11(10):2214-2220. doi: 10.1111/j.1600-6143.2011.03685 . . .
14. Parikh C R, Hall I E, Bhangoo R S, et al. Associations of Perfusate Biomarkers and Pump Parameters With Delayed Graft Function and Deceased Donor Kidney Allograft Function. Am J Transplant. 2016; 16(5):1526-1539. doi: 10.1111/ajt.13655.
15. Arykbaeva A S, Vries D K de, Doppenberg J B, et al. Metabolic needs of the kidney graft undergoing normothermic machine perfusion. Kidney International. 2021; 0(0). doi: 10.1016/j.kint.2021.04.001.
16. De Deken J, Kocabayoglu P, Moers C. Hypothermic machine perfusion in kidney transplantation. Current Opinion in Organ Transplantation. 2016; 21(3):294-300. doi: 10.1097/MOT.0000000000000306.
17. Guy A J, Nath J, Cobbold M, et al. Metabolomic Analysis of Perfusate During Hypothermic Machine Perfusion of Human Cadaveric Kidneys. Transplantation. 2015; 99(4):754-759. doi: 10.1097/TP.0000000000000398.
18. Hall I E, Akalin E, Bromberg J S, et al. Deceased-donor acute kidney injury is not associated with kidney allograft failure. Kidney Int. 1; 95(1): 199-209. doi: 10.1016/j.kint.2018.08.047.
19. Hall I E, Schröppel B, Doshi M D, et al. Associations of deceased donor kidney injury with kidney discard and function after transplantation. Am J Transplant. 2015; 15(6):1623-1631. doi: 10.1111/ajt. 13144.
20. Reese P P, Hall I E, Weng F L, et al. Associations between Deceased-Donor Urine Injury Biomarkers and Kidney Transplant Outcomes. J Am Soc Nephrol. 2016; 27(5):1534-1543. doi: 10.1681/ASN.2015040345.
21. Evans A M, DeHaven C D, Barrett T, Mitchell M, Milgram E. Integrated, nontargeted ultrahigh performance liquid chromatography/electrospray ionization tandem mass spectrometry platform for the identification and relative quantification of the small-molecule complement of biological systems. Anal Chem. 2009; 81(16):6656-6667. doi: 10.1021/ac901536h.
22. Hall I E, Reese P P, Doshi M D, et al. Delayed Graft Function Phenotypes and 12-Month Kidney Transplant Outcomes. Transplantation. 8; 101(8):1913-1923. doi: 10.1097/TP.0000000000001409.
23. OPTN. A guide to calculating and interpreting the Kidney Donor Profile Index (KDPI). Published online 2018.
24. Reese P P, Harhay M N, Abt P L, Levine M H, Halpern S D. New Solutions to Reduce Discard of Kidneys Donated for Transplantation. JASN. 2016; 27(4):973-980. doi: 10.1681/ASN.2015010023.
25. Stallone G, Grandaliano G. To discard or not to discard: transplantation and the art of scoring. Clinical Kidney Journal. 2019; 12(4):564-568. doi: 10.1093/ckj/sfz032.
26. Kosieradzki M, Rowiński W. Ischemia/reperfusion injury in kidney transplantation: mechanisms and prevention. Transplant Proc. 2008; 40(10):3279-3288. doi: 10.1016/j.transproceed.2008.10.004.
27. Bonventre J V, Yang L. Cellular pathophysiology of ischemic acute kidney injury. J Clin Invest. 2011; 121(11):4210-4221. doi: 10.1172/JCI45161.
28. Annema W, Dikkers A, de Boer J F, et al. HDL Cholesterol Efflux Predicts Graft Failure in Renal Transplant Recipients. J Am Soc Nephrol. 2016; 27(2):595-603. doi: 10.1681/ASN.2014090857.
29. Castelló IB. Hyperlipidemia: A risk factor for chronic allograft dysfunction. Kidney International. 2002; 61: S73-S77. doi: 10.1046/j.1523-1755.61.s80.13.
30. Wu N, Yang M, Gaur U, Xu H, Yao Y, Li D. Alpha-Ketoglutarate: Physiological Functions and Applications. Biomol Ther (Seoul). 2016; 24(1):1-8. doi: 10.4062/biomolther.2015.078.
31. Jeppsson A, Ekroth R, Friberg P, et al. Renal effects of alpha-ketoglutarate early after coronary operations. Ann Thorac Surg. 1998; 65(3):684-690. doi: 10.1016/s0003-4975(97)01337-4.
32. Bienholz A, Petrat F, Wenzel P, et al. Adverse effects of α-ketoglutarate/malate in a rat model of acute kidney injury. American Journal of Physiology-Renal Physiology. 2012; 303(1):F56-F63. doi: 10.1152/ajprenal.00070.2012.
33. Sonkar K, Ayyappan V, Tressler C M, et al. Focus on the glycerophosphocholine pathway in choline phospholipid metabolism of cancer. NMR Biomed. 2019; 32(10):e4112. doi: 10.1002/nbm.4112.
34. Liu Y, Yan S, Ji C, et al. Metabolomic Changes and Protective Effect of L-Carnitine in Rat Kidney Ischemia/Reperfusion Injury. KBR. 2012; 35(5):373-381. doi: 10.1159/000336171.
35. Gallazzini M, Burg M B. What's New About Osmotic Regulation of Glycerophosphocholine. Physiology (Bethesda). 2009; 24:245-249. doi: 10.1152/physiol.00009.2009.
36. Sis B, Sarioglu S, Celik A, et al. Renal medullary changes in renal allograft recipients with raised serum creatinine. J Clin Pathol. 2006; 59(4):377-381. doi: 10.1136/jcp.2005.029181.
37. Strifler G, Tuboly E, Görbe A, Boros M, Pécz D, Hartmann P. Targeting Mitochondrial Dysfunction with L-Alpha Glycerylphosphorylcholine. PLOS ONE. 2016; 11(11):e0166682. doi: 10.1371/journal.pone.0166682.
38. Duranton F, Cohen G, De Smet R, et al. Normal and pathologic concentrations of uremic toxins. J Am Soc Nephrol. 2012; 23(7):1258-1270. doi: 10.1681/ASN.2011121175.
39. Niwa T. Organic acids and the uremic syndrome: protein metabolite hypothesis in the progression of chronic renal failure. Semin Nephrol. 1996; 16(3):167-182.
40. Niwa T. Recent progress in the analysis of uremic toxins by mass spectrometry. J Chromatogr B Analyt Technol Biomed Life Sci. 2009; 877(25):2600-2606. doi: 10.1016/j.jchromb.2008.11.032.
41. Kopple J D. Phenylalanine and tyrosine metabolism in chronic kidney failure. J Nutr. 2007; 137(6 Suppl 1):1586S-1590S; discussion 1597S-1598S. doi: 10.1093/jn/137.6.1586S.
42. Boirie Y, Albright R, Bigelow M, Nair K S. Impairment of phenylalanine conversion to tyrosine inend-stage renal disease causing tyrosine deficiency. Kidney International. 2004; 66(2):591-596. doi: 10.1111/j.1523-1755.2004.00778.
43. Møller N, Meek S, Bigelow M, Andrews J, Nair K S. The kidney is an important site for in vivo phenylalanine-to-tyrosine conversion in adult humans: A metabolic role of the kidney. PNAS. 2000; 97(3):1242-1246. doi: 10.1073/pnas.97.3.1242.

### SUPPLEMENTAL MATERIALS AND METHODS

Machine perfusion. Further detail about the LifePort Kidney Transporter machine perfusion device can be found in the operator's manual.3 Blank sample solutions tested included SPS-1 and KPS-1 solutions (Organ Recovery Systems, Itasca, IL), which include manufacturer-stated compositions.^{1,2}

KPS-1 (constituents in amount/1000 ml): calcium chloride (dihydrate) (0.068 g); sodium hydroxide (0.70 g); HEPES (free acid) (2.38 g); potassium phosphate (monobasic) (3.4 g); mannitol (USP) (5.4 g); glucose, beta D (+) (1.80 g); sodium gluconate (17.45 g); magnesium gluconate D (−) gluconic acid, hemimagnesium salt (1.13 g); ribose, D (−) (0.75 g); hydroxyethyl Starch (HES) (50.0 g); glutathione (reduced form) (0.92 g); and adenine (free base) (0.68 g).

SPS-1 (constituents in amount/1000 ml): hydroxyethyl starch (HES) (50 g); lactobionic acid (as Lactone) (35.83 g); potatssium phosphate monobasic (3.4 g); magnesium sulfate heptahydrate (1.23 g); raffinose pentahydrate (17.83 g); adenosine (1.34 g); allopurinol (0.136 g); glutathione (reduced form) (0.922 g); and potassium hydroxide (5.61 g)

Perfusate measurements. Metabolite measurements underwent the Metabolon scaling and imputation process, in which each metabolite's measurement units were scaled such that 1 unit was equivalent to the median of the detectable sample measurements. Missing values were then imputed to the minimum detected level among the samples for each metabolite. The instrumental variability, quantified by the internal standards added to each sample before injection into the mass spectrometers, had a median coefficient of variation (% CV) of 5, as provided by Metabolon.

Statistics. % CVs were calculated from a set of split samples in the study and the perfusate solution duplicates. % CVs for each metabolite were calculated as the average across all duplicate pairs of the SD divided by the mean, for all pairs for which the metabolite was detected and quantified. Split sample identity was verified through hierarchical clustering and correlation comparisons.

Among paired kidneys, the present inventor evaluated the Spearman correlation of post-perfusate metabolites between all paired kidneys, and all combinations of non-paired kidneys. To explore the correlation of metabolites between paired kidneys after accounting for perfusion time differences, the present inventor performed linear regressions for all continuous metabolites of the metabolite levels of the left kidney as a function of levels of the right kidney, adjusted for the L/R pair perfusion time differences.

The present inventor used agglomerative clustering to observe structure in post-HMP perfusate data using a Manhattan distance dissimilarity matrix and Ward's linkage. MAD-scaled continuous metabolites detected in at least 50% of samples were used as features. Kidneys were considered to have been paired in the dendrogram when a cluster of 2 leaves was formed by the left and right kidneys of the same donors.

In a supplemental analysis, the present inventor additionally adjusted for the concentration of metabolite measured in baseline perfusate. To evaluate if the association between metabolite and dcGF varied with high-risk donors, models with and interaction term of the metabolite and high-risk donor definitions were examined. DCD donors (yes vs no) and by KDPI (≥80 vs <80) were explored. To estimate the DGF OR per MAD of metabolite measurement, the present inventor fit logistic regression models adjusted for perfusion time and then additionally for KDPI. To evaluate the significance of metabolite change versus perfusion time, unadjusted regression between the post:base ratio of metabolite and perfusion time was used. The present inventor evaluated the significance of the dcGF and DGF association of each de novo metabolite, then adjusted the significance of all de novo metabolite associations with outcome for false discovery. Pathways were analyzed using the fold change of median metabolite measurement in dcGF vs non-dcGF kidneys fordcGF-associated de novo metabolites using Ingenuity Pathway Analysis (IPA) to characterize pathways differentially active in dcGF kidneys. All analyses were completed in R 4.0.3.

### SUPPLEMENTAL REFERENCES

1. KPS-1 Kidney Perfusion Solution. Organ Recovery Systems. Accessed Oct. 21, 2022. https://www.organ-recovery.com/preservation-solutions/kps-1-kidney-perfusion-solution/.
2. SPS-1 (UW Solution) Solution. Organ Recovery Systems. Accessed Oct. 21, 2022. https://www.organ-recovery.com/preservation-solutions/sps-1-static-preservation-solution/.
3. 755-00002_Rev_K_LKT101_Operators_Manual.pdf. Accessed Oct. 21, 2022. https://www.organ-recovery.com/wp-content/uploads/2019/07/755-00002_Rev_K_LKT101_Operators_Manual.pdf.

TABLE 1
Donor and recipient characteristics

| Deceased donor characteristics | | (N = 147) | Recipient and transplant characteristics | | (N = 190) |
|---|---|---|---|---|---|
| Age, y | | 47 + 14 | Age, y | | 57 + 14 |
| Male | | 93 (63%) | Male | | 124 (65%) |
| Black race | | 26 (18%) | Black race | | 86 (45%) |
| Hispanic ethnicity | | 28 (19%) | Hispanic ethnicity | | 21 (11%) |
| BMI, kg/m² | | 29 ⊥ 8 | BMI, kg/m2 | | 29 ⊥ 6 |
| Hypertension | | 64 (44%) | Cause ESRD | Diabetes | 23 (12%) |
| Diabetes | | 16 (11%) | | Hypertension | 70 (37%) |
| Cause of death | Head Trauma | 28 (19.0%) | | Glomerulonephritis | 52 (27%) |
| | Anoxia | 55 (37.4%) | | Graft failure | 26 (14%) |
| | Stroke | 60 (40.8%) | | Other or unknown | 19 (10%) |
| | Other | 4 (2.7%) | Preemptive transplant | | 0 (0%) |
| Hepatitis C seropositive | | 0 (0%) | Dialysis duration, | | 58 ± 37 |
| | | | months | | |
| ECD | | 42 (29%) | Pre-transplantation blood transfusion | | 46 (24%) |
| DCD | | 27 (18%) | PRA | 0% | 136 (71.6%) |
| KDRI | | 1.42 ⊥ 0.43 | | 1%-20% | 12 (6.3%) |
| KDPI, % | | 58 ± 26 | | 21%-80% | 18 (9.5%) |
| KDPI ≥ 80 | | 36(24%) | | >80% | 24 (12.6%) |
| Admission to procurement, days | | 7 ± 5 | Cold ischemia time, h | | 17.9 ± 7.3 |
| Admission SCr, mg/dL | | 1.11 ± 0.41 | Pump duration, h | | 9.9 ± 5.7 |
| Terminal SCr, mg/dL | | 1.44 ± 1.22 | HLA mismatch | 0 | 2 (1%) |
| Urine creatinine, mg/dL | | 59.26 ± 55.70 | | 1 | 2 (1%) |
| Number of kidneys discarded | 0 | 130 (88%) | | 2 | 6 (3%) |
| | 1 | 17 (12%) | | 3 | 19 (10%) |
| Donor AKI stage | No AKI | 106 (72%) | | 4 | 50 (26%) |
| | Stage 1 | 23 (16%) | | 5 | 77 (41%) |
| | Stage 2 | 7 (5%) | | 6 | 34 (18%) |
| | Stage 3 | 11 (7%) | | | |

Values are mean ± SD or n (%)
AKI, acute kidney injury; BMI, body mass index; DCD, donation after cardiovascular determination of death; ECD, expanded-criteria donor; ESRD, end-stage renal disease; KDPI, kidney donor profile index; KDRI, kidney donor risk index; PRA, panel reactive antibody; SCr, serum creatinine

TABLE 2
Post-HMP perfusate metabolite association with dcGF. These 6 metabolites were significantly associated
with dcGF after adjusting for pump time and correcting for false discovery. The dcGF HRs after
adjusting for KDPI and baseline perfusate are also shown.

| | | | | dcGF HR per MAD (95% CI) | |
|---|---|---|---|---|---|
| | Detected in | Mean Fold | | +Pump Time | +Pump Time and |
| Metabolite | samples, % | Elevation in dcGF | Sub-pathway | Adjusted* | KDPI Adjusted |
| Alpha-ketoglutarate | 99% | 1.53 | TCA cycle | 1.45 (1.17-1.79) | 1.36 (1.10-1.68) |
| 1-Palmitoyl-2- | 98% | 1.57 | Phosphatidylcholine | 1.29 (1.13-1.47) | 1.28 (1.07-1.51) |
| docosahexaenoyl-GPC | | | | | |
| (16:0/22:6) | | | | | |
| 1-Palmitoyl-2-dihomo- | 97% | 1.60 | Phosphatidylcholine | 1.17 (1.07-1.27) | 1.18 (1.06-1.31) |
| linolenoyl-GPC (16:0/20:3n3 or | | | | | |
| 6) | | | | | |
| 1-Stearoyl-2-docosahexaenoyl- | 85% | 1.65 | Phosphatidylcholine | 1.28 (1.11-1.47) | 1.25 (1.04-1.51) |
| GPC (18:0/22:6) | | | | | |
| 1-Carboxyethylphenylalanine | 83% | 1.87 | Phenylalanine | 1.04 (1.02-1.05) | 1.03 (1.01-1.04) |
| | | | metabolism | | |
| 3-Carboxy-4-methyl-5-propyl- | 74% | 1.97 | Fatty acid- | 1.15 (1.06-1.24) | 1.10 (0.99-1.22) |
| 2-furanpropanoate (CMPF) | | | dicarboxylate | | |

*The 6 dcGF-associated metabolites, discussed in the results and FIG. 3, were selected using the pump time-adjusted model

TABLE 3
dcGF-associated functional pathways. P value of pathway overlap
in Ingenuity Pathway Analysis is the likelihood that the
overlap between significant molecules observed and
a given pathway is due to random chance.

| | P Value of | Observed |
|---|---|---|
| Functional pathway | pathway Overlap | Changed Molecules |
| Cell death and | 2.92E−06 | pyruvic acid, |
| survival | | quinolinic acid |
| Concentration of | 6.23E−06 | 2-oxoglutaric acid, |
| fatty acids | | arachidonic acid, |
| | | docosahexaenoic acid, |
| | | N(1)-methylnicotinamide, |
| | | pyruvic acid, |
| | | quinolinic acid |
| Transport of amino | 1.04E−05 | glutathione disulfide, |
| acids | | piperidine, |
| | | pyruvic acid, |
| | | quinolinic acid, |
| | | spermine |
| Production of reactive | 3.53E−05 | alpha-ketoglutarate, |
| oxygen species | | CMPF, |
| | | arachidonic acid, |
| | | docosahexaenoic acid, |
| | | pyruvic acid, |
| | | quinolinic acid |
| Concentration of | 8.61E−04 | arachidonic acid, |
| glutathione | | docosahexaenoic acid, |
| | | glutathione disulfide, |
| | | pyruvic acid |

TABLE 4
Deceased donor characteristics for kidneys that were discarded

| | | | Discarded Kidney | |
|---|---|---|---|---|
| Discarded kidney donor characteristics | | Overall (N = 32) | Characteristics | (N = 35) |
| Age, y | | 53 ± 14 | Perfusion time, h | 10.9 ± 5.6 |
| Male | | 20 (63%) | UNOS discard reason: | |
| Black race | | 5 (16%) | No recipient located | 12 (34%) |
| Hispanic ethnicity | | 3 (9%) | Biopsy findings | 8 (23%) |
| BMI, kg/m² | | 32 ± 8 | Anatomical abnormalities | 4 (11%) |
| Hypertension | | 15 (47%) | Poor organ function | 2 (6%) |
| Diabetes | | 3 (9%) | Organ not as described | 1 (3%) |
| Cause of death | Head trauma | 9 (28%) | Vascular damage | 1 (3%) |
| | Anoxia | 9 (28%) | Other | 7 (20%) |
| | Stroke | 13 (41%) | | |
| | Other | 1 (3%) | | |
| Hepatitis C seropositive | | 1 (3%) | | |
| ECD | | 14 (44%) | | |
| DCD | | 8 (25%) | | |
| KDRI | | 1.63 ± 0.47 | | |
| KDPI, % | | 72 ± 25 | | |
| Admission to procurement, days | | 6 ± 6 | | |
| Admission SCr, mg/dL | | 1.05 ± 0.38 | | |
| Terminal SCr, mg/dL | | 1.58 ± 1.00 | | |
| Urine creatinine, mg/dL | | 76.45 ± 60.09 | | |
| Number of kidneys discarded | 1 | 13 (41%) | | |
| | 2 | 19 (59%) | | |
| Donor AKI stage | No AKI | 17 (53%) | | |
| | Stage 1 | 10 (31%) | | |
| | Stage 2 | 2 (6%) | | |
| | Stage 3 | 3 (9%) | | |

Values are mean ± SD or n (%)
AKI, acute kidney injury; BMI, body mass index; DCD, donation after cardiovascular determination of death; ECD, expanded-criteria donor; KDPI, kidney donor profile index; KDRI, kidney donor risk index; SCr, serum creatinine

TABLE 5
dcGF associations of KDPI > 80 and expanded criteria donor subgroups. Significant interaction terms (P < 0.10)
with high KDPI were observed for 2 metabolites and with ECD for 1 metabolite

| | Overall | | DCD | Non-DCD | | KDPI ≥ 80 | KDPI < 80 |
|---|---|---|---|---|---|---|---|
| | (n = 190) | | (n = 39) | (n = 151) | | (n = 47) | (n = 143) |
| | Time- | | Time- | Time- | High | Time- | Time- |
| | adjusted | DCD | adjusted | adjusted | KDPI | adjusted | adjusted |
| | dcGF HR | interaction | dcGF HR | dcGF HR | interaction | dcGF HR | dcGF HR |
| Biochemical | (95% CI) | p | (95% CI) | (95% CI) | p | (95% CI) | (95% CI) |
| alpha-ketoglutarate | 1.45 (1.17- | 0.030* | 1.69 (1.25, | 1.24 (1.01, | 0.817 | 1.26 (1.04, | 1.36 (0.92, |
| | 1.79) | | 2.31) | 1.54) | | 1.53) | 2.02) |
| 1-palmitoyl-2-docosahexaenoyl- | 1.29 (1.13- | 0.053 | 1.52 (1.17, | 1.19 (1.04, | 0.099 | 0.93 (0.62, | 1.33 (1.18, |
| GPC (16:0/22:6) | 1.47) | | 1.97) | 1.35) | | 1.41) | 1.50) |
| 1-palmitoyl-2-dihomo-linolenoyl- | 1.17 (1.07- | 0.030* | 1.23 (1.12, | 0.97 (0.77, | 0.694 | 1.07 (0.75, | 1.18 (1.11, |
| GPC (16:0/20:3n3 or 6)* | 1.27) | | 1.34) | 1.21) | | 1.51) | 1.26) |
| 1-stearoyl-2-docosahexaenoyl- | 1.28 (1.11- | 0.042* | 1.66 (1.12, | 1.16(1.03, | 0.124 | 0.91 (0.60, | 1.30 (1.14, |
| GPC (18:0/22:6) | 1.47) | | 2.47) | 1.30) | | 1.39) | 1.48) |
| 1-carboxyethylphenylalanine | 1.04 (1.02- | 0.007* | 1.46 (1.06, | 1.03 (1.01, | 0.874 | 1.02 (1.01, | 1.01 (0.78, |
| | 1.05) | | 2.02) | 1.05) | | 1.04) | 1.31) |
| 3-carboxy-4-methyl-5-propyl-2- | 1.15 (1.06- | 0.775 | 1.06 (0.87, | 1.11 (1.05, | 0.096 | 1.02 (0.94, | 1.14 (1.07, |
| furanpropanoate (CMPF) | 1.24) | | 1.28) | 1.18) | | 1.12) | 1.23) |

*Significant interaction; interaction term p-value < 0.05

TABLE 6
deGF associations after adjustment for baseline perfusate

| | | dcGF HR per MAD (95% CI), | |
|---|---|---|---|
| | | Adjusted for Baseline Perfusate Metabolite | |
| | | +Pump Time | +pump time and KDPI |
| Metabolite | Sub-pathway | Adjusted | adjusted |
| Alpha-ketoglutarate | TCA cycle | 1.39 (1.19-1.62) | 1.33 (1.13-1.57) |
| 1-Palmitoyl-2-docosahexaenoyl-GPC (16:0/22:6) | Phosphatidylcholine | 1.17 (1.11-1.22) | 1.16 (1.09-1.24) |
| 1-Palmitoyl-2-dihomo-linolenoyl-GPC (16:0/20:3n3 or 6)* | Phosphatidylcholine | 1.35 (1.20-1.52) | 1.30 (1.11-1.53) |
| 1-Stearoyl-2-docosahexaenoyl-GPC (18:0/22:6) | Phosphatidylcholine | 1.34 (1.14-1.58) | 1.27 (1.01-1.59) |
| 1-Carboxyethylphenylalanine | Phenylalanine metabolism | 1.15 (0.91-1.44) | 1.05 (0.79-1.40) |
| 3-Carboxy-4-methyl-5-propyl-2-furanpropanoate (CMPF) | Fatty acid dicarboxylate | 1.10 (1.02-1.19) | 1.03 (0.93-1.15) |

TABLE 7
Association of delayed graft function with deGF-associated metabolites

| | | | OR for DGF per MAD (95% CI) | |
|---|---|---|---|---|
| | Detected in % | | Perfusion Time- | Perfusion Time + KDPI- |
| Metabolite | of Samples | Sub-pathway | Adjusted | Adjusted |
| Alpha-ketoglutarate | 99% | TCA cycle | 1.30 (1.02-1.66) | 1.25 (0.97-1.61) |
| 1-Palmitoyl-2-docosahexaenoyl-GPC | 98% | Phosphatidylcholine | 1.30 (1.02-1.64) | 1.19 (0.95-1.50) |
| (16:0/22:6) | | | | |
| 1-Palmitoyl-2-dihomo-linolenoyl-GPC | 97% | Phosphatidylcholine | 1.20 (0.98-1.49) | 1.16 (0.94-1.44) |
| (16:0/20:3n3 or 6)* | | | | |
| 1-Stearoyl-2-docosahexaenoyl-GPC | 85% | Phosphatidylcholine | 1.29 (1.01-1.66) | 1.17 (0.93-1.49) |
| (18:0/22:6) | | | | |
| 1-Carboxyethylphenylalanine | 83% | Phenylalanine | 1.18 (0.96-1.45) | 1.14 (0.93-1.41) |
| | | metabolism | | |
| 3-Carboxy-4-methyl-5-propyl-2- | 74% | Fatty acid, dicarboxylate | 1.15 (1.00-1.32) | 1.10 (0.96-1.26) |
| furanpropanoate (CMPF) | | | | |

TABLE 8
Comparison of metabolite values between kidneys that were discarded, transplanted, had dcGF, and did not have dcGF.
After Bonferonni adjustment, no significant differences in metabolite levels between groups were found.
Bonferroni-adjusted P value: 0.05/54 = 0.00093; Post short for post-HMP, base short for baseline. Biochemical values
are in relative units provided by Metabolon, presented as median (IQR). Wilcoxon rank-sum test used.

| | | | | | | P Value | P Value | P Value |
|---|---|---|---|---|---|---|---|---|
| | | Discarded | Transplanted | deGF | No deGF | (discard v. | (discard | (discard vs |
| Biochemical | Timepoint | (n=35) | (n = 190) | (n =33) | (n = 157) | transplant) | v. dcGF) | no dcGF) |
| 1-Carboxyethylphenyl- | Basc | 0.19 | 0.19 | 0.19 | 0.19 | 0.8576 | 0.7029 | 0.7516 |
| alanine | | (0.19-0.74) | (0.19-0.74) | (0.19-0.53) | (0.19-0.76) | | | |
| | Post | 1.00 | 0.93 | 0.93 | 0.92 | 0.6331 | 0.6580 | 0.6561 |
| | | (0.40-1.88) | (0.40-1.72) | (0.42-1.33) | (0.40-1.74) | | | |
| | Post: Base | 1.74 | 1.82 | 2.19 | 1.75 | 0.9244 | 0.6055 | 0.7978 |
| | | (1.00-5.36) | (1.05-3.74) | (1.08-3.70) | (1.05-3.75) | | | |
| 1-Palmitoyl-2- | Base | 0.31 | 0.37 | 0.44 | 0.37 | 0.6056 | 0.9701 | 0.5473 |
| dihomo-linolenoyl- | | (0.04-0.73) | (0.04-0.85) | (0.04-0.93) | (0.09-0.82) | | | |
| GPC (16:0/20:3n3 or 6)* | Post | 1.47 | 1.21 | 1.40 | 1.16 | 0.5176 | 0.8443 | 0.4089 |
| | | (0.73-2.28) | (0.76-1.98) | (0.90-1.99) | (0.75-1.97) | | | |
| | Post: Base | 2.58 | 3.07 | 4.31 | 2.57 | 0.9448 | 0.2589 | 0.8217 |
| | | (1.30-17.36) | (1.48-11.39) | (2.46-17.58) | (1.41-10.40) | | | |
| 1-Palmitoyl-2- | Base | 0.26 | 0.33 | 0.40 | 0.33 | 0.3576 | 0.3516 | 0.4010 |
| docosahexaenoyl- | | (0.10-0.76) | (0.10-0.85) | (0.10-1.25) | (0.10-0.82) | | | |
| GPC (16:0/22:6) | Post | 1.60 | 1.19 | 1.47 | 1.15 | 0.4053 | 0.4419 | 0.2285 |
| | | (0.86-2.08) | (0.69-2.00) | (0.93-2.71) | (0.64-1.95) | | | |
| | Post: Base | 3.05 | 3.07 | 3.63 | 2.87 | 0.2050 | 0.9514 | 0.1267 |
| | | (1.68-9.02) | (1.42-6.23) | (2.13-8.90) | (1.39-6.16) | | | |
| 1-Stearoyl-2- | Base | 0.16 | 0.16 | 0.16 | 0.16 | 0.7861 | 0.5509 | 0.8734 |
| docosahexaenoyl- | | (0.16-0.54) | (0.16-0.61) | (0.16-0.63) | (0.16-0.53) | | | |
| GPC (18:0/22:6) | Post | 0.95 | 0.94 | 1.10 | 0.83 | 0.2454 | 0.5640 | 0.1227 |
| | | (0.63-2.00) | (0.49-1.68) | (0.81-1.99) | (0.46-1.67) | | | |
| | Post: Base | 3.57 | 2.72 | 4.07 | 2.56 | 0.2868 | 0.7219 | 0.1714 |
| | | (1.64-5.79) | (1.26-5.73) | (1.74-7.96) | (1.23-5.47) | | | |
| 3-Carboxy-4-methyl- | Base | 0.13 | 0.13 | 0.34 | 0.13 | 0.6149 | 0.1442 | 0.8489 |
| 5-propyl-2- | | (0.13-0.37) | (0.13-0.53) | (0.13-0.84) | (0.13-0.46) | | | |
| furanpropanoate (CMPF) | Post | 1.26 | 0.62 | 1.07 | 0.52 | 0.0518 | 1.0000 | 0.0203 |
| | | (0.30-2.86) | (0.13-1.41) | (0.45-1.99) | (0.13-1.22) | | | |
| | Post: Base | 2.37 | 1.68 | 2.13 | 1.57 | 0.0231 | 0.3126 | 0.0151 |
| | | (1.39-9.26) | (1.00-4.33) | (1.26-5.57) | (1.00-4.26) | | | |
| Alpha-ketoglutarate | Base | 0.67 | 0.52 | 0.31 | 0.57 | 0.7471 | 0.2762 | 0.4944 |
| | | (0.14-1.53) | (0.15-1.60) | (0.12-0.79) | (0.20-1.63) | | | |
| | Post | 1.12 | 1.06 | 1.27 | 1.06 | 0.7144 | 0.5395 | 0.5449 |
| | | (0.69-2.11) | (0.61-1.81) | (0.73-2.21) | (0.52-1.68) | | | |
| | Post: Base | 1.78 | 1.51 | 5.72 | 1.19 | 0.7851 | 0.1450 | 0.4675 |
| | | (0.63-10.54) | (0.54-7.93) | (1.16-32.32) | (0.46-6.10) | | | |

1. A method for assessing viability of a donor organ undergoing perfusion comprising the steps of:
(a) measuring levels of a panel of metabolites comprising one or more of alpha-ketoglutarate (AKG), 3-carboxy-4-methyl-5-propyl-2-furanpropanoate (CMPF), 1-carboxyethylphenylalanine, 1-palmitoyl-2-docosahexaenoyl-glycerophosphocholine (GPC), 1-palmitoyl-2-dihomo-linolenoyl-GPC, and 1-stearoyl-2-docosahexaenoyl-GPC, from a first sample obtained from the organ at the beginning of perfusion; and
(b) measuring levels of the panel of metabolites from a second sample obtained from the organ at or near the end of perfusion,
wherein levels of the metabolites above a reference indicates that the donor organ is not viable, and wherein levels of the metabolites below the reference indicates that the donor organ is viable.

2. The method of claim 1, wherein the donor organ comprises a liver, kidney, heart, pancreas, small intestine, limb, extremity, or a portion of any of the foregoing.

3. The method of claim 1, wherein the sample comprises perfusion solution from the organ, biopsy from the organ or a fluid produced by the organ.

4. The method of claim 1, wherein the beginning of perfusion comprises within about ten minutes of the start of perfusion.

5. The method of claim 1, wherein at or near the end of perfusion comprises just prior to transfer of the donor organ from an organ procurement organization (OPO) to the recipient medical center.

6. The method of claim 1, wherein at or near the end of perfusion comprises just after transfer of the donor organ from an OPO to the recipient medical center.

7. A method for identifying a donor organ as viable or not viable for transplantation comprising the steps of:
(a) measuring levels of a panel of metabolites comprising one or more of AKG, CMPF, 1-carboxyethylphenylalanine, 1-palmitoyl-2-docosahexaenoyl-GPC, 1-palmitoyl-2-dihomo-linolenoyl-GPC, and 1-stearoyl-2-docosahexaenoyl-GPC, from a sample obtained from an ex vivo organ undergoing perfusion;
(b) comparing the measured metabolite levels with the levels of the same metabolites in a reference or control that indicate organ dysfunction or function; and
(c) identifying the donor organ as viable when the measured metabolite levels are below the reference levels and identifying the donor organ as not viable when the measured metabolite levels are above the reference levels.

8. The method of claim 7, wherein the donor organ comprises a liver, kidney, heart, pancreas, small intestine, limb, extremity, or a portion of any of the foregoing.

9. The method of claim 7, wherein the sample comprises perfusion solution from the organ, biopsy from the organ or a fluid produced by the organ.

10. A method for transplanting a donor organ that has undergone perfusion comprising the steps of:
(a) measuring levels of a panel of metabolites comprising one or more of AKG, CMPF, 1-carboxyethylphenylalanine, 1-palmitoyl-2-docosahexaenoyl-GPC, 1-palmitoyl-2-dihomo-linolenoyl-GPC, and 1-stearoyl-2-docosahexaenoyl-GPC, from a sample obtained from an ex vivo organ undergoing perfusion;
(b) transplanting the donor organ to a recipient when the measured levels are below a reference level of the metabolites that indicates organ dysfunction.

11. The method of claim 10, wherein the donor organ comprises a liver, kidney, heart, pancreas, small intestine, limb, extremity, or a portion of any of the foregoing.

12. The method of claim 10, wherein the sample comprises perfusion solution from the organ, biopsy from the organ or a fluid produced by the organ.
